(19) United States
(12) Patent Application Publication
(10) Pub. No.: US 2007/0009570 A1
(43) Pub. Date: Jan. 11, 2007

(54) Method for preparing porous polymer scaffold for tissue engineering using gel spinning molding technique

(76) Inventors: Sang Heon Kim, Seoul (KR); Soo Hyun Kim, Seoul (KR); Young Ha Kim, Seoul (KR); Jae Hyun Kwon, Seoul (KR); Min Sub Chung, Seoul (KR)

Correspondence Address: JONES DAY, 222 EAST 41ST ST, NEW YORK, NY, 10017, US

(21) Appl. No.: 11/482,393

(22) Filed: Jul. 6, 2006

(30) Foreign Application Priority Data
Jul. 7, 2005 (KR) 10-2005-61080
Apr. 17, 2006 (KR) 10-2006-34400

(51) Int. Cl. A61F 2/00; B28B 1/08

(52) U.S. Cl. 424/423; 264/69

(57) ABSTRACT

The present invention relates to a method of preparing a porous polymer scaffold for tissue engineering using a gel spinning molding technique. The method of the present invention can prepare a porous polymer scaffold having a uniform pore size, high interconnectivity between pores and mechanical strength, as well as high cell seeding and proliferation efficiencies, which can be effectively used in tissue engineering applications. Further, the method of the present invention can easily mold a porous polymer scaffold in various types such as a tube type favorable for regeneration of blood vessels, esophagus, nerves and the like, as well as a sheet type favorable for regeneration of skins, muscles and the like, by regulating the shape and size of a template shaft.

### FIELD OF THE INVENTION

The present invention relates to a method for preparing a porous polymer scaffold for tissue engineering using a gel spinning molding technique. In particular, the present invention relates to a method for preparing a porous polymer scaffold having a high interconnectivity between pores and an optimal mechanical strength. The porous polymer scaffold prepared according to the method of the present invention shows high cell seeding and proliferation efficiencies. Thus, the porous polymer scaffold of the present invention can be effectively used in tissue engineering applications.

### BACKGROUND OF THE INVENTION

Polymers have been widely used in biomedical applications. Especially, polymers have been used to develop biodegradable and biocompatible raw materials, which can be used to fabricate scaffolds for purposes of tissue regeneration.

Scaffolds for tissue engineering have to satisfy the following requirements: 1) good biocompatibility without any occurrences of transplant rejection, cytotoxicity and inflammatory reaction; 2) high cell seeding and proliferation efficiencies; 3) uniform pore size and high porosity to facilitate material transportation; 4) high interconnectivity between pores; and 5) mechanical strength sufficient to endure in vivo pressure.

There are various methods for preparing a porous polymer scaffold, some examples of which are as follows: a solvent-casting/particle-leaching method (Mikos, et al., Polymer, 35: 1068, 1994); a gas foaming method (Harris, et al., J. Biomed. Mater. Res., 42: 396, 1998); a gas foaming salt method (Nam, et al., J. Biomed. Mater. Res., 53: 1, 2000); a fiber extrusion and fabric forming process (Paige, et al., Tissue Engineering, 1: 97, 1995); a liquid-liquid phase separation method (Schugens, et al., J. Biomed. Mater. Res., 30: 449, 1996); an emulsion freeze-drying method (Whang, et al., Polymer, 36: 837, 1995); and an electrospinning method (Matthews, et al., Biomacromolecules, 3: 232, 2002).

However, the scaffolds prepared by the above methods have many problems when being used for biological tissue engineering, which is performed to induce three-dimensional tissue regeneration via the adhesion and proliferation of cells.

For example, a sponge-type scaffold, which is prepared by the solvent-casting/particle-leaching method or the gas foaming salt method, shows desirable pore sizes and high porosity. However, its mechanical strength is extremely weak. In addition, a fiber-type scaffold prepared by the electrospinning method shows high porosity, but its pore sizes are too small to achieve a three-dimensional cell culture.

Further, there have been numerous attempts in the art to prepare a nonwoven-type scaffold by a melt spinning method using a biodegradable aliphatic polyester such as polyglycolic acd (PGA), poly(lactic acid-co-glycolic acid) (PLGA) and the like. However, the mechanical strength of the nonwoven-type scaffold prepared by such method is also too low for use in tissue engineering applications. In order to maintain a desired shape, such scaffold has been processed to induce bonding between fibers by soaking it in a polylactic acid (PLA) solution prepared by dissolving PLA in an organic solvent such as methylenechloride, pulling it out from the solution, removing the residual PLA solution therefrom and drying it in an oven. However, since numerous conditions have to consider such as selecting a proper solvent according to the type of polymer used, temperature control, compatibility between polymers, etc., such process is very complicated and difficult to use.

### SUMMARY OF THE INVENTION

The object of the present invention is to provide a method of preparing a porous polymer scaffold having an uniform pore size, high interconnectivity between pores, high cell seeding and proliferation efficiencies and superior mechanical strength. The porous polymer scaffold of the present invention is adapted to be effectively used in tissue engineering applications.

In accordance with one aspect of the present invention, there is provided a method of preparing a porous polymer scaffold, which comprises the following steps:

(i) preparing a polymer solution by dissolving a biocompatible polymer in an organic solvent;

(ii) spinning the polymer solution prepared in the step (i) into a non-solvent being stirred by a shaft under rotation so as to form a polymer gel;

(iii) winding the polymer gel formed in the step (ii) around the shaft under rotation to mold a porous polymer scaffold; and

(iv) drying the porous polymer scaffold obtained in the step (iii) to remove the organic solvent therefrom.

In accordance with another aspect of the present invention, there is provided a porous polymer scaffold prepared according to said method, which has a pore size ranging from 1 to 800 microns and a porosity ranging from 40 to 99%.

### BRIEF DESCRIPTION OF DRAWINGS

FIG. 1 is a schematic diagram showing the preparation of a porous polymer scaffold of the present invention by using a gel spinning molding technique.

FIG. 2 is a schematic diagram of a gel spinning molding device constructed in accordance with the present invention.

FIG. 3 is a photograph showing a tube type PLCL porous polymer scaffold prepared in Example 1.

FIG. 4 is a scanning electron microscopy (SEM) photograph showing the surface of a tube type PLCL porous polymer scaffold prepared in Example 1 (40× magnification).

FIG. 5 is a SEM photograph showing the surface of a tube type PLCL porous polymer scaffold prepared in Example 1 (200× magnification).

FIG. 6 is a SEM photograph showing the cross-section of a tube type PLCL porous polymer scaffold prepared in Example 1 (40× magnification).

FIG. 7 is a SEM photograph showing the cross-section of a tube type PLCL porous polymer scaffold prepared in Example 1 (200× magnification).

FIG. 8 is a photograph showing a sheet type PLLA porous polymer scaffold prepared in Example 2.

FIG. 9 is a SEM photograph showing the surface of a sheet type PLLA porous polymer scaffold prepared in Example 2 (40× magnification).

FIG. 10 is a graph showing the cell seeding efficiencies of PLCL porous polymer scaffolds prepared in Example 1 and Comparative Example 1.

### DETAILED DESCRIPTION OF THE PREFERRED EMBODIMENTS

The method of preparing a porous polymer scaffold of the present invention is characterized by the following steps: spinning a polymer fiber into a non-solvent being stirred by a template shaft; phase-separating the spun polymer fiber into a polymer gel; and winding the polymer gel around the template shaft simultaneously with the phase-separation to mold the porous polymer scaffold.

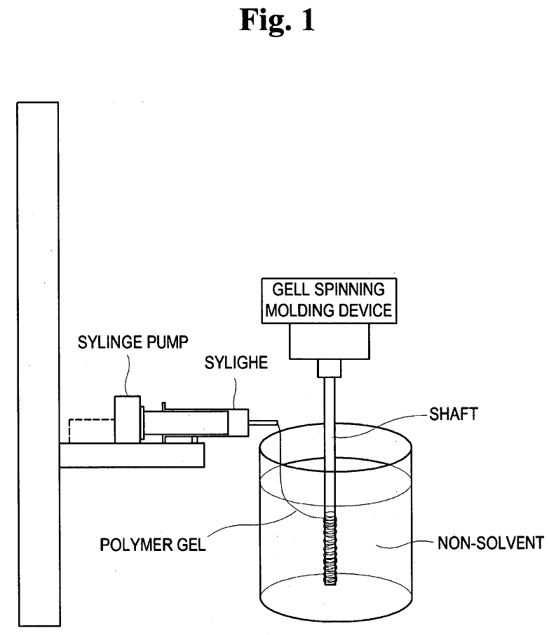

The preferred embodiment of preparing a porous polymer scaffold of the present invention by using a gel spinning molding technique is described in FIG. 1.

In particular, a molding device is installed to sufficiently soak a shaft in a non-solvent, wherein the shaft is then rotated. A polymer solution is prepared by dissolving a biodegradable polymer in an organic solvent and subjecting it to falling-spinning in the non-solvent being stirred by the shaft rotated at a rate of 5 to 50 Ml/min through the use of a spinning nozzle such as a syringe. The polymer solution, which is spun in the non-solvent, undergoes phase-separation into gel-state fibers. Then, simultaneously with the phase-separation, the gel-state fibers wind around the shaft used as a template under rotation at a uniform orbit. At this time, adhesion occurs between the wound fibers, which results in the molding of a porous polymer scaffold. It is preferred to employ the polymer solution at a concentration ranging from 1 to 20% based on weight to volume ratio (w/v). Subsequently, the porous polymer scaffold is dried at an ambient temperature or under vacuum in order to completely remove the residual organic solvent.

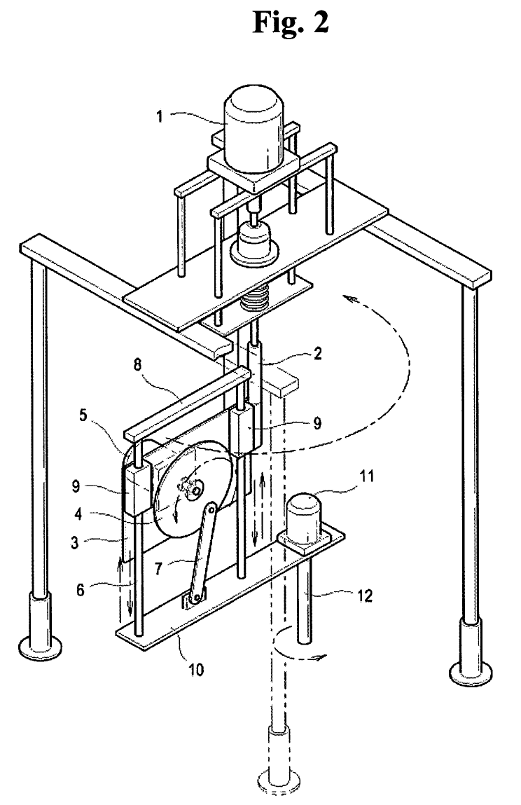

In order to perform the method of the present invention, it is possible to employ a gel spinning molding device equipped with a revolution driving device, a rotation driving device, a up-and-down driving device and a shaft capable of operating in revolution, rotation and up-and-down motions by the action of said devices (shown in FIG. 2).

In particular, the molding device comprises: a revolution driver (1) vertically located at the uppermost part of an installation surface; a revolution driving device having a principal axis (2) connected to the revolution driver (1); a first connecting plate (3) linked to the principal axis (2) and being configured to rotate therewith; a rotating plate (4) installed at the first connecting plate (3) and being configured for rotation; a up-and-down driver (5) rotating the rotating plate (4); a up-and-down driving device having a sub-arm (7) connected to the rotating plate (4); a second connecting plate (10) connecting the sub-arm (7) to a rotation driver (11); a pair of vertical connecting stands (6), which are connected to the upper side of the second connecting plate (10), glidingly extended by penetrating a connecting groove (9) of the first connecting plate (3) and being fixed by a horizontal fixed stand (8) at the upper part; a rotation driving device having the rotation driver (11) installed at the second connecting plate (10); and a shaft (12) linked to the rotation driver (11).

The shaft (12) is operated to move in a revolution motion on the principal axis (2) through the action of the revolution driving device. Further, the shaft (12) can move in a rotation motion through the action of the rotation driving device and can also move up-and-down by the action of the up-and-down driving device. It is preferred to operate the template shaft in revolution, rotation and up-and-down motions at the rate of 50 to 300 rpm, 50 to 500 rpm or 50 to 300 rpm.

When employing the molding device of the present invention, the template shaft can perform all ranges of motion, i.e., revolution, rotation and up-and-down. Thus, the spun fibers can evenly wind around the shaft without leaning to one particular side thereof.

Further, since the gel spinning molding device of the present invention can independently regulate the rates of the revolution, rotation and up-and-down drivers by using three separated motors, it can control the rate and direction of winding a gel-phase polymer fiber around the template shaft by properly regulating the rate of each driver. Further, the gel spinning molding device of the present invention can also prepare a porous polymer scaffold having a suitable shape by regulating the shape, size and thickness of the shaft. For example, a tube type scaffold can be prepared by employing a cylindrical shaft and a sheet type scaffold, by employing a reel-shaped shaft. Additionally, the tube's diameter and the sheet's size can be regulated by properly controlling the diameter of the cylindrical shaft and the reel-shaped shaft, respectively.

The polymers, which can be employed in the present invention, include biocompatible polymers not subject to any transplant rejection, inflammatory reaction and cytotoxicity, e.g., biodegradable or non-degradable synthetic polymers, natural polymers, copolymers and mixtures thereof. Since the density, structure of pores and porosity of a porous polymer scaffold are influenced by the type and molecular weight of a polymer used for the preparation thereof, it is preferable to select a polymer that is adapted for the intended purpose of the porous polymer scaffold. There is no limitation on the molecular weight of the polymer used, although it is preferable to use a polymer having a weight mean molecular weight (M_{w} ) ranging from 5,000 to 1,000,000. Since a polymer having a molecular weight deviating from such range shows a viscosity that is too low or too high, it is difficult to control the pore size and porosity of a fiber. In particular, a polymer having a molecular weight of less than 5,000 shows such a weak mechanical strength that it cannot be used as a biomaterial.

The biodegradable synthetic polymers include, but are not limited to, poly(L-lactic acid) (PLLA), poly(D,L-lactic acid) (PDLA) , polyglycolic acid (PGA) , polycarprolactone (PCL) , polytrimethylene carbonate , polydioxanone, polyhydroxyalkanoate (PHA), polyorthoester , polyhydroxyester, polyprophylene fumarate, polyphosphazene, polyanhydride and the like. The non-degradable synthetic polymers include, but are not limited to, polyurethane, polyethylene, polycarbonate, polyethylene oxide and the like. The biodegradable natural polymers include, but are not limited to, collagen, fibrin, chitosan, hyaluronic acid, cellulose, polyamino acid, fibroin, sericin and a derivative thereof.

Further, in addition to the use of a single polymer, the following can be employed: a copolymer consisting of 2 or more types of monomers, e.g., poly(lactic acid-co-glycolic acid) (PLGA), poly(L-lactic acid-co-caprolactone) (PLCL), etc.; or a mixture of 2 or more types of polymers, e.g., a mixture comprising a synthetic polymer selected from the group consisting of PLLA, PDLA, PGA, PLGA and the like and a natural polymer such as collagen.

The organic solvents used for dissolving said polymer include, but are not limited to, chloroform, methylene chloride, acetic acid, ethylacetate, dimethylcarbonate, tetrahydrofuran and a mixture thereof.

When a polymer solution is spun into a non-solvent, the gel-state polymer fiber has to be coagulated at a proper rate in a non-solvent, thereby making it possible to obtain a homogenous porous polymer scaffold with good interconnectivity. Therefore, it is preferable to employ a non-solvent, which is easy to mix with the organic solvent used for dissolving a polymer and allows the phase-separation of a spun polymer into a gel state at a proper rate. The non-solvents employable in the present invention include, but are not limited to, water, methanol, ethanol, hexane, heptane and mixtures thereof.

The method of preparing a porous polymer scaffold according to the present invention can regulate the characteristics of a porous polymer scaffold by controlling the types of polymer, organic solvent and non-solvent, as well as by controlling the concentration and spinning rate of polymer solution, rotation rate of a shaft and the like. For example, the lower the concentration of a polymer solution, the higher the porosity and interconnectivity between the pores of a porous polymer scaffold become. Further, the higher the concentration of a polymer solution, the stronger the mechanical strength of a porous polymer scaffold becomes. Also, it is possible to regulate the winding rate and direction of a polymer fiber around the shaft by controlling the spinning rate of a polymer solution and the rotation rate of a shaft, thereby facilitating the regulation of pore characteristics and mechanical strength of a porous polymer scaffold. Considering all the factors described above, it is preferred that the pore size of a porous polymer scaffold is in the range from 1 to 800 microns, while the porosity thereof is in the range from about 40 to 99%. Accordingly, the method of the present invention can prepare a porous polymer scaffold by regulating the pore size and porosity according to its intended purpose.

According to the method of the present invention, the polymer solution is spun in the non-solvent when the spun polymer fibers are molded into a porous polymer scaffold, which simplifies the preparation process. Further, the method of the present invention has the advantage of facilitating the preparation of a porpus polymer scaffold having a desired shape and size by regulating the shape and size of a template shaft.

Various modifications are possible in the preparation process of the present invention. For example, it is possible to prepare a porous polymer scaffold with a multilayer structure, which is constructed by spinning heterologous polymers having a different constitution and arrangement at regular intervals.

According to the above-described method of the present invention, the phase-separated polymer fibers are wound around the shaft under rotation, while adhesion occurs at various spots of the fibers. Thus, there is a strong interaction between the fibers, thereby leading to the porous polymer scaffold with strong mechanical strength.

Further, since the porous polymer scaffold, which is prepared according to the method of the present invention, has a three-dimensional structure of pores (uniform in size and interconnected with each other without any separation), it displays high cell seeding and proliferation efficiencies and facilitates the diffusion of biologically active substances through the pores. Therefore, the porous polymer scaffold of the present invention can be effectively used for cell culture and tissue regeneration.

Accordingly, the porous polymer scaffold, which is prepared according to the present invention, can be effectively used as a raw material for fabricating artificial tissues or organs such as artificial blood vessels, artificial esophagus, artificial nerves, artificial hearts, prostatic heart valves, artificial skins, artificial muscles, artificial ligaments, artificial respiratory organs, etc. Further, the porous polymer scaffold of the present invention can be prepared in the form of a hybrid tissue with functional cells derived from tissues or organs. It may have various biomedical applications, for example, to maintain cell functions, tissue regenerations, etc.

The present invention will now be described in detail with reference to the following examples, which are not intended to limit the scope of the present invention.

### EXAMPLE 1

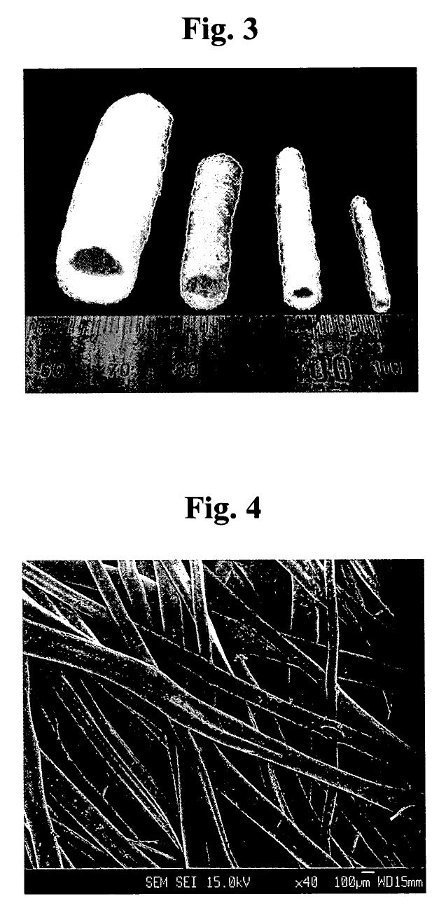

A polymer solution was prepared by dissolving PLCL (the composition rate of monomers=50:50) having a weight mean molecular weight (M_{w}) of 340,000 in chloroform at a final concentration of 10% w/v. It was then poured in a syringe. A molding device (shown in FIG. 2) was installed at a container having 5 L of mixed solvent of methanol and hexane (“non-solvent”) to soak a shaft in the non-solvent. The shaft was then operated to perform rotation, revolution and up-and-down motions at a rate of 100 rpm, 150 rpm and 100 rpm, respectively. At this time, four different types of cylindrical shafts having diameters of 10, 6, 5 and 2 mm, respectively, were employed. The polymer solution in the syringe was subjected to falling spinning at a rate of 10 Ml/min with a syringe pump in the non-solvent, which is in rotation due to the shaft. The spun polymer solution was phase-separated into polymer gel fibers. Simultaneously with the phase-separation, the polymer gel fibers wind around the shaft, which is operated to perform revolution, rotation and up-and-down motions in the non-solvent to form a porous polymer scaffold. The porous polymer scaffolds, which are formed as a result, were then dried in a vacuum oven to completely remove the residual organic solvent. As such, four different types of tube type porous polymer scaffolds having diameters of 10, 6, 5 and 2 mm, respectively, and a thickness of 1 mm were obtained (shown in FIG. 3).

The diameter of each fiber constituting the scaffolds prepared above ranges from 40 to 100 microns, while its pore size ranges from 50 to 150 microns. Further, its porosity, which was measured with a mercury injection pore measuring instrument, ranges from about 60 to 70%. In order to examine the mechanical properties of the scaffolds, the tensile strength, tensile modulus and elastic constant were measured while pulling 500 neuton (N) of a load cell along a cylindrical direction of the scaffold at a rate of 100 mm/min using an Instron. The results obtained therefrom were described in Table 1. A restoring force of the porous polymer scaffold was maintained over 98% when it was pulled up to 400% of its original length.

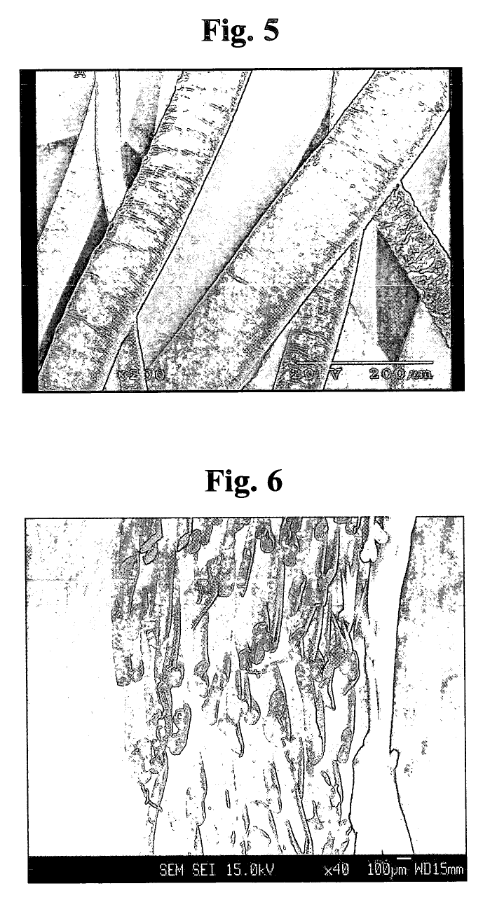

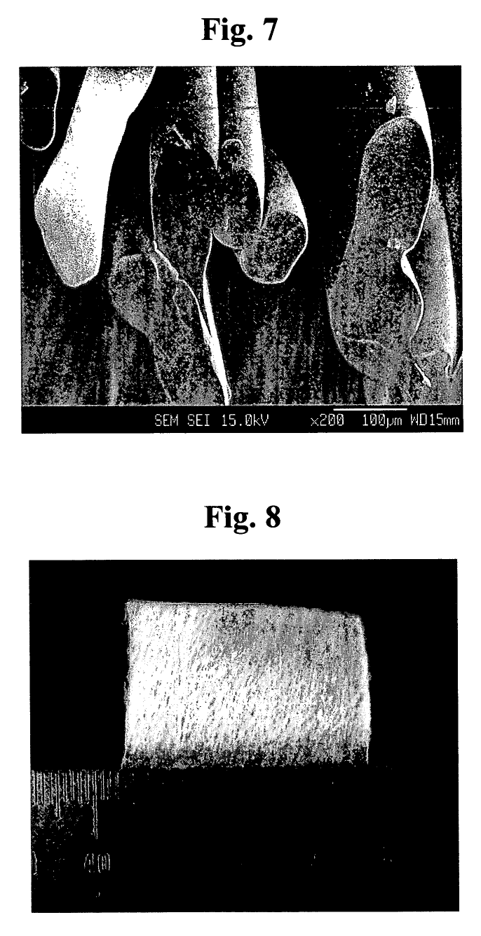

Further, the surface and cross-section of the porous polymer scaffold, which was prepared according to the method of the present invention, was observed with a scanning electron microscope (SEM), as shown in FIG. 4 (surface; 40× magnification), FIG. 5 (surface; 200× magnification), FIG. 6 (cross-section; 40× magnification) and FIG. 7 (cross-section; 200× magnification). As a result, it was confirmed that the porous polymer scaffold of the present invention is composed of properly adhered fibers. It was further confirmed that such scaffold shows high interconnectivity between pores and has a uniform pore size.

### EXAMPLE 2

The porous polymer scaffold was prepared according to the same method as described in Example 1, except that a polymer solution was prepared by dissolving PLLA having a weight mean molecular weight (M_{w}) of 150,000 in chloroform at a final concentration of 5% w/v, methanol was employed as a non-solvent and a reel-shaped shaft was employed. As a result, the sheet type porous polymer scaffold having 32 mm in width and in length and and a thickness of 2 mm was prepared, as shown in FIG. 8.

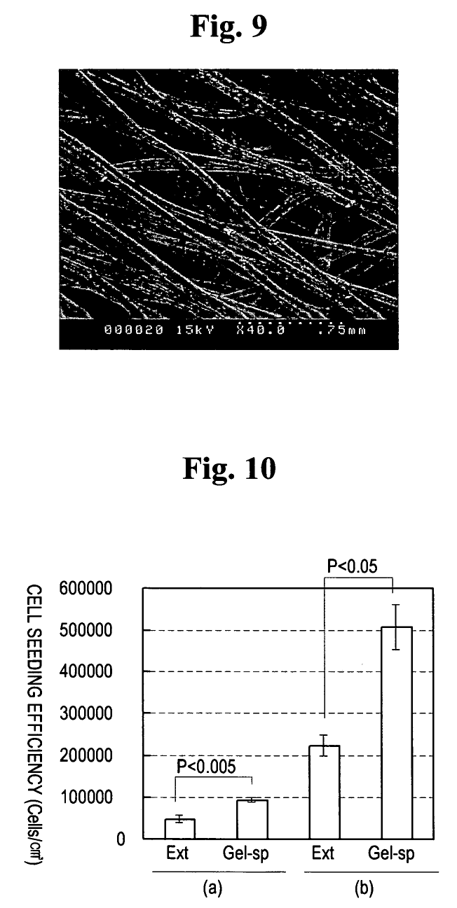

The diameter of each fiber constituting the porous polymer scaffold prepared above ranges from 50 to 100 microns, while its pore size ranges from 50 to 150 microns. Further, its porosity, which was measured by a mercury injection pore measuring instrument, ranges from about 60 to 70%. Also, the surface of the porous polymer scaffold was observed with a SEM. As can be seen from FIG. 9 (40× magnification), it was confirmed that the porous polymer scaffold of the present invention is composed of properly adhered fibers. Moreover, such scaffold shows high interconnectivity between pores and has a uniform pore size.

### COMPARATIVE EXAMPLE 1

A polymer solution was prepared by dissolving PLCL (50:50) having a weight mean molecular weight (M_{w}) of 340,000 in chloroform at a final concentration of 20% w/v. Sodium chloride having a particle size ranging from 100 to 200 microns was added to the polymer solution so as to adjust the weight ratio of sodium chloride/PLCL to 90 wt % and then homogenously mixed with a voltex mixer. The prepared polymer solution was subjected to extrusion molding with an extruder and then completely dried for 7 days. The resulting sample was soaked in distilled water to entirely elute sodium chloride remaining within the sample and freeze-dried so as to obtain a porous polymer scaffold.

The mechanical properties of the porous polymer scaffold, which was prepared by the gel spinning molding method as described in Example 1, were compared with those of the porous polymer scaffold prepared by the extrusion molding method as described in Comparative Example 1. All the samples used for the comparison were 0.5 cm in length and 2 cm in width.

TABLE 1

| | | Tensile | | Tensile |
|---|---|---|---|---|
| | Elastic | strength | Thickness of | modulus |
| | constant (%) | (MPa) | scaffold (mm) | (MPa) |
| Example 1 | 534 | 4.50 | 0.96 | 1.376 |
| Comparative | 442 | 1.17 | 0.94 | 0.232 |
| Example 1 | | | | |

As can be seen from Table 1, it was confirmed that the porous polymer scaffold, which was prepared according to the method of the present invention (Example 1), shows about 4-fold higher tensile strength than the scaffold prepared according to the extrusion molding method (Comparative Example 1).

### TEST EXAMPLE 1

### Cell Seeding Efficiency

The compatibilities of the cell cultures of porous polymer scaffolds prepared in Example 1 and Comparative Example 1 were observed as follows.

Smooth muscle cells of rabbit were isolated according to an enzyme method (Michael et al., In vitro Cell. Dev. Biol., 39: 402, 2003) and each of the porous polymer scaffolds were seeded with the isolated cells. The cell seeding efficiency was measured by analyzing the cell survival activity with WST-8(2-(2-methoxy-4-nitrophenyl)-3-(4-nitrophenyl)-5-(2,4-disulfophenyl)-2H-tetrazolium, monosodium salt).

The cell survival activity was measured at two different cell concentrations, i.e., a high concentration of 3.5×10⁶ cells/cm³ (a) and a low concentration of 3.5×10⁵ cells/cm³ (b), respectively. The results are provided in FIG. 10. In FIG. 10, Ext means the cell seeding efficiency of the porous polymer scaffold prepared by the extrusion molding method (Comparative Example 1), while Gel-sp means the cell seeding efficiency of the porous polymer scaffold prepared by the gel spinning molding method of the present invention (Example 1). As a result, it has been confirmed that the porous polymer scaffold, which was prepared according to the method of the present invention (Example 1), shows about 2- to 3-fold higher cell seeding efficiency than the polymer scaffold prepared by the extrusion molding method (Comparative Example 1).

While the present invention has been described and illustrated with respect to a preferred embodiment of the invention, it will be apparent to those skilled in the art that variations and modifications are possible without deviating from the broad principles and teachings of the present invention, which should be limited solely by the scope of the claims appended hereto.

What is claimed is:

1. A method of preparing a porous polymer scaffold, comprising the steps of:
(i) preparing a polymer solution by dissolving a biocompatible polymer in an organic solvent;
(ii) spinning the polymer solution prepared in the step (i) in a non-solvent stirred by a rotating shaft to form a polymer gel;
(iii) winding the polymer gel formed in the step (ii) around the rotating shaft to mold a porous polymer scaffold; and
(iv) drying the porous polymer scaffold obtained in the step (iii) to remove the organic solvent therefrom.

2. The method of claim 1, wherein the step (ii) of forming the polymer gel is simultaneously conducted with the step (iii) of molding the porous polymer scaffold.

3. The method of claim 1, wherein the biocompatible polymer is selected from the group consisting of biodegradable synthetic polymer, non-degradable synthetic polymer, biodegradable natural polymer, copolymers and mixtures thereof.

4. The method of claim 3, wherein the biodegradable synthetic polymer is selected from the group consisting of poly(L-lactic acid), poly(D,L-lactic acid), polyglycolic acid (PGA) , polycarprolactone (PCL) , polytrimethylene carbonate , polydioxanone, polyhydroxyalkanoate, polyorthoester, polyhydroxyester, polyprophylene fumarate, polyphosphazene, polyanhydride, copolymers and mixtures thereof.

5. The method of claim 3, wherein the non-degradable synthetic polymer is selected from the group consisting of polyurethane, polyethylene, polycarbonate, polyethyleneoxide, copolymers and mixtures thereof.

6. The method of claim 3, wherein the biodegradable natural polymer is selected from the group consisting of collagen, fibrin, chitosan, hyaluronic acid, cellulose, polyamino acid, fibroin, cerisin, copolymers and mixtures thereof.

7. The method of claim 1, wherein the organic solvent is selected from the group consisting of chloroform, methylene chloride, acetic acid, ethylacetate, dimethylcarbonate, tetrahydrofuran and mixtures thereof.

8. The method of claim 1, wherein the non-solvent is selected from the group consisting of water, methanol, ethanol, hexane, heptane and mixtures thereof.

9. The method of claim 1, wherein the shaft performs revolution and rotation motions while moving up-and-down.

10. A porous polymer scaffold prepared according to the method of claim 1 having a pore size ranging from 1 to 800 microns and porosity ranging from 40 to 99%.
